(19) United States
(12) Patent Application Publication
(10) Pub. No.: US 2020/0246191 A1
(43) Pub. Date: Aug. 6, 2020

(54) NEGATIVE PRESSURE WOUND DRESSING AND NEGATIVE PRESSURE WOUND TREATMENT DEVICE

(71) Applicant: EVOPHANCIE BIOTECH LTD, New Taipei City (TW)

(72) Inventors: Chi-Hsiang Lu, New Taipei City (TW); Jun-Wei Hong, New Taipei City (TW); Shang-Ru Lin, New Taipei City (TW)

(21) Appl. No.: 16/777,950

(22) Filed: Jan. 31, 2020

(30) Foreign Application Priority Data
Feb. 1, 2019 (TW) 108201730

(51) Int. Cl. A61F 13/00; A61L 15/16

(52) U.S. Cl. CPC A61F 13/00068; A61F 13/00012; A61F 13/00029; A61L 2300/41; A61L 2300/406; A61L 2300/414; A61L 15/16

(57) ABSTRACT

The present application provides a negative pressure wound dressing, including foam and a bio-cellulose film with a three-dimensional reticular structure formed on a surface of the foam, wherein a projected area of the bio-cellulose film on a bonding surface of the foam and the bio-cellulose film is greater than or equal to that of the foam. The presence of the bio-cellulose film between the foam and a wound avoids the foam from sticking to the wound and the growth of new tissue into the foam when performing a negative pressure wound therapy. Also, when replacing the foam, a secondary damage to the wound is reduced, so as to relieve pain that the patient suffers.

### BACKGROUND

### 1. Technical Field

The present application relates to a wound dressing, and more particularly to a wound dressing for a negative pressure wound therapy.

### 2. Description of the Related Art

Negative Pressure Wound Therapy (NPWT) is an auxiliary treatment for wound healing. It is suitable for many acute and chronic wounds, including orthopedics, soft tissues, skin grafts, pressure sores, lower extremity venous ulcers, diabetic foot, surgery infection, postoperative wounds, etc. The NPWT is placed in a cleaned wound with a foam corresponding to a shape of the wound, and the foam is in full contact with the wound surface. Since there are many holes in the foam, a vacuum pump can be used to apply a negative pressure to the wound filled with the foam. The pressure causes pus and infectious substances in the wound to be discharged through the holes in the foam, and also attracts the tissue fluid into the foam to maintain the moist therapy environment of the wound, so as to promote blood microcirculation around the wound and the growth of the new tissue. Thus, the effect of accelerating wound healing is achieved.

However, the foam in NPWT, especially those using hydrophobic polyurethane foam, has high adhesion to the wound, and the new tissue easily grows into the holes in the foam, and the foam must be replaced about every two to three days. If the infection of the wound is serious, the frequency of replacing the foam may be more according to the physician's diagnosis. Every time the foam is changed, due to the adhesion of the wound and the growth of the new tissue, patients often suffer severe pain, and there is also a secondary injury to the wound, which is a serious problem in clinical application.

Therefore, how to avoid the adhesion of foam to the wound and to avoid the growth of new tissue into the holes in the foam is keenly aimed to be achieved in the clinical and industrial fields.

### SUMMARY

To solve the above problems, the present application provides a negative pressure wound dressing comprising foam and a bio-cellulose film, the bio-cellulose film having a three-dimensional reticular structure and bonded to the surface of the foam, wherein a projected area of the bio-cellulose film on the surface of the form bonded to the bio-cellulose film is greater than or equal to that of the foam.

The bio-cellulose film refers to a microorganism, for example, bacteria of at least one genus selected from the group consisting of Gluconacetobacter, Acetobacter, Rhizobium, Sarcina, Pseudomonas, Achromobacter, Alcaligenes, Enterobacter, Azotobacter and Agrobacterium, which uses the carbon source in the culture medium to form β-1,4-glucan chain in the cell, and then secrets bio-cellulose extracellularly to form the bio-cellulose film.

In one embodiment, the bio-cellulose film is formed by culturing bacteria in a culture medium having a carbon source, a nitrogen source, and, optionally, a yeast extract. The culture is, for example, a static culture. The carbon source includes, for example, glucose, mannitol, molasses, fructose, sucrose, etc. The nitrogen source is such as peptone. Further, a weight ratio of the carbon source, the nitrogen source, and the yeast extract may be 60:6:6 to 15:3:0; or 60:6:6 to 15:3:0.5; or 60:6:6 to 15:3:1; or 60:6:6 to 15:3:2; or 60:6:6 to 15:3:3; or 60:6:6 to 15:3:4; or 60:6:6 to 15:3:5.

In one embodiment, the three-dimensional reticular structure has a plurality of backbone fibers parallel to each other and a plurality of interlayered fibers interwoven with any two adjacent backbone fibers, wherein the plurality of backbone fibers and the plurality of interlayered fibers are interwoven with each other to form nano-sized pores. In another embodiment, the backbone fibers and the interlayered fibers are made of bio-celluloses, and a diameter of the backbone fiber is greater than or equal to a diameter of the interlayered fiber.

In one embodiment, the bio-cellulose has a diameter of 20 to 100 nm.

In one embodiment, the bio-cellulose film has a bio-cellulose content of 0.0013 to 0.0018 g/cm² per unit area.

In one embodiment, the negative pressure wound dressing comprises a drug. For example, the drug for promoting wound healing, e.g., antibacterial or anti-inflammatory, and promotion of cell growth, includes but is not limited to, at least one selected from the group consisting of antibiotics, antibacterial agents, growth factors, fibrin, vasodilators, angiogenesis promoters, antioxidants, and anti-inflammatory agents. More specifically, the drug is contained in the foam, in the bio-cellulose film, or in both of the foam and the bio-cellulose film.

In one embodiment, the foam comprises at least one material selected from the group consisting of polyurethane foam, polyvinyl alcohol foam, polyether foam, polyester foam, polylactic acid foam, polyolefin foam, chitosan foam, cellulose foam, alginate foam, gelatin foam, and collagen foam.

In another embodiment, the foam has a multilayer structure, including, e.g., a contact layer, an absorption layer and a drainage layer, wherein the contact layer is in contact with the bio-cellulose film.

The present application also provides a negative pressure wound treatment device, comprising: the negative pressure wound dressing; a liquid delivery tube connected to the negative pressure wound dressing; and a negative pressure source.

In one embodiment, the negative pressure wound treatment device further comprises a sealing film, which is disposed on the negative pressure wound dressing and is used to cover a wound and a normal skin outside the wound.

In another embodiment, an opening is provided in the center of the sealing film and penetrates through two opposite surfaces of the sealing film, and one end of the liquid delivery tube extends through the opening to the foam of the negative pressure wound dressing.

In one embodiment, the negative pressure wound treatment device further comprises a liquid-collecting tank connected to the other end of the liquid delivery tube, and the liquid-collecting tank is connected to the negative pressure source through a connecting tube.

The negative pressure wound dressing of the present application is bonded to the surface of the foam through the bio-cellulose film. When performing the negative pressure wound treatment (NPWT), the bio-cellulose film exists between the foam filled in the wound and the wound surface. When changing the dressing, it can avoid the wound adhering to the material, and reduce the severe pain in the patient. Further, since the bio-cellulose film has a three-dimensional reticular structure and the three-dimensional reticular structure has nano-sized pores formed by interweaving backbone fibers and interlayered fibers, it has excellent gas permeability, and does not damage the negative pressure environment. On the other hand, the nano-sized pores of the bio-cellulose film and the pores of the foam can also carry drugs, which can release the drug on the surface of the wound while sucking out the pus and the infectious substance, so as to promote the healing of the wound.

### BRIEF DESCRIPTION OF THE DRAWINGS

FIG. 1 is a schematic diagram showing the structure of the negative pressure wound dressing in the present application;

FIGS. 2A and 2B are scanning electron micrographs (SEM) of the bio-cellulose film of the negative pressure wound dressing in the present application, wherein FIG. 2A is a photograph showing a three-dimensional reticular structure of a bio-cellulose film, and FIG. 2B is a photograph showing a side view of the bio-cellulose film;

FIG. 3 is a schematic view showing the foam of the negative pressure wound dressing in the present application having a multilayer structure; and

FIGS. 4A to 4D are schematic diagrams for implementing the negative pressure wound treatment device in the present application.

### DETAILED DESCRIPTION OF THE EMBODIMENTS

The examples of the present application are described below by way of embodiments, and those skilled in the art can readily appreciate the advantages and functions of the present application from the disclosure herein. The present application may be embodied or applied by other different embodiments. The details of the present specification may also be based on different opinions and applications, and various modifications and changes may be made without departing from the spirit of the present application. In addition, all ranges and values herein are inclusive and combinable. Any value or point falling within the ranges recited herein, such as any integer, may be the minimum or maximum value to derive the lower range and the like.

In the preparation of the negative pressure wound dressing in the present application, the foam is placed on a bottom surface or fixed in a container with a culture medium, and the liquid level of the culture medium is slightly higher than the foam. The foam is completely immersed in the culture medium, and statically culturing bacteria of at least one genus selected from the group consisting of genus Gluconacetobacter, Acetobacter, Rhizobium, Sarcina, Pseudomonas, Achromobacter, Alcaligenes, Enterobacter, Azotobacter and Agrobacterium in the culture medium, preferably, bacteria of one single genus such as genus Gluconacetobacter or Acetobacter. The culture time is preferably 24 to 96 hours. After the culture, the bio-cellulose film is formed on the foam, and the bio-cellulose film is bonded to the surface of the foam, wherein the culture medium includes a carbon source, a nitrogen source, and a yeast extract.

The projected area of the foam on the horizontal plane needs to be less than or equal to the solution surface area of the culture medium, so that the projected area of the bio-cellulose film, which formed by the gas-liquid interface, on the surface where the foam is bonded to the bio-cellulose film is greater than or equal to the projected area of the foam.

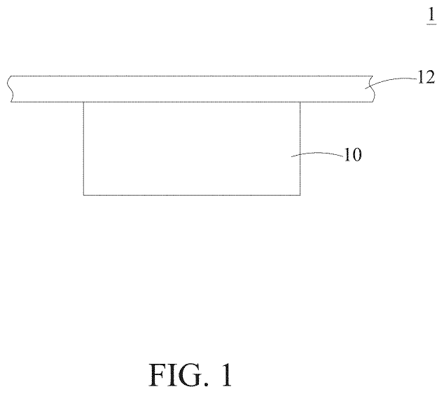

As shown in FIG. 1, the negative pressure wound dressing 1 of the present application comprises a foam 10; and a bio-cellulose film 12 disposed on the foam 10 to bond the bio-cellulose film 12 to the surface of the foam 10. Further, the projected area of the bio-cellulose film 12 on the surface where the foam 10 is bonded to the bio-cellulose film 12 is larger than the projected area of the foam 10. In another embodiment, the projected area of the bio-cellulose film 12 on the surface where the foam 10 is bonded to the bio-cellulose film 12 may be equal to the projected area of the foam 10.

In one embodiment, the culture medium comprises mannitol as a carbon source, peptone as a nitrogen source, and, optionally, a yeast extract, in a ratio by weight of 60:6:6 to 15:3:0. The culture medium is controlled to be acidic, for example, with a pH of between 0.5 and 6.5, the absorbance value reflecting the microbial concentration in the culture medium (wavelength setting of 620 nm) is controlled in the range of between 0.006 to 0.01, culture temperature is between 25 to 28° C., and the culture time is 24 to 96 hours. After testing, the bio-cellulose film has a thickness of at least 0.3 to 0.6 mm, and the bio-cellulose in the bio-cellulose film has a diameter of about 20 to 100 nm. Further, the bio-cellulose film has a bio-cellulose content of from 0.0013 to 0.0018 g/cm² per unit area.

The static culture refers to the bacteria form a bio-cellulose in a non-woven manner on the surface of the culture medium; the bio-cellulose accumulates and extends from the surface of the culture medium toward the bottom of the container; then accumulated bio-cellulose gradually touches the surface of the foam; and finally, the bio-cellulose grows into the foam, such that the bio-cellulose film is bonded with the foam.

In addition, the container for static culture is preferably a flat container, which means that a height of the container is lower relative to a length and width of the container; through a lower container height and a wider growth area, the oxygen consumption of bacteria is controlled, so as to regulate the diameter of the bio-cellulose.

In one embodiment, the bio-cellulose film is formed by statically culturing bacteria of the genus Gluconacetobacter in a culture medium having mannitol, peptone, and yeast extract for fermentation.

The bacteria of the genus Gluconacetobacter are preferably Gluconacetobacter xylinum, which is characterized in that it is easy to control the degree of the thickness of the obtained bio-cellulose.

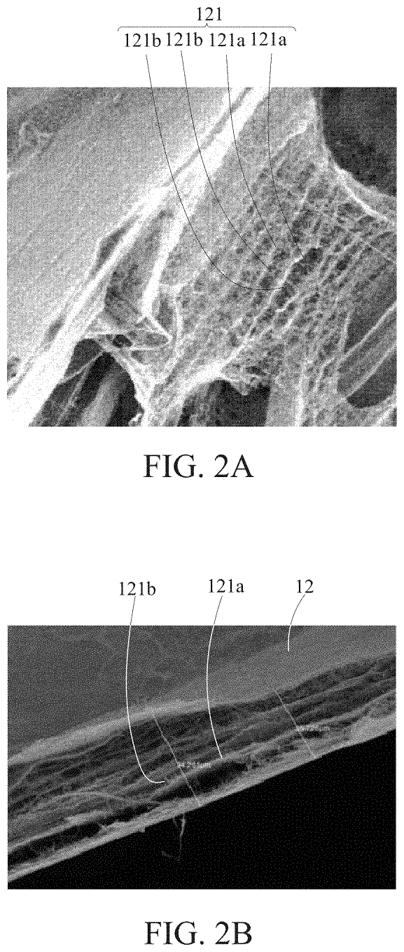

In one embodiment, the bio-cellulose film of the negative pressure wound dressing in the present application, which has a three-dimensional reticular structure, is as shown in FIG. 2A. The three-dimensional reticular structure 121 is formed by a plurality of bio-celluloses. Specifically, the three-dimensional reticular structure 121 has a plurality of backbone fibers 121a extending parallel to each other, or extending in the longitudinal direction or the width direction of the bio-cellulose film, and interlayered fibers 121b interwoven with the backbone fibers 121a. Thus, the interlayered fibers 121b are in the horizontal direction and in the vertical direction connected to any two adjacent backbone fibers 121a, so as to form a three-dimensional reticular structure 121. The backbone fibers 121a and the interlayered fibers 121b are all bio-cellulose. As shown, the diameter of the backbone fiber 121a is greater than or equal to the diameter of the interlayered fiber 121b. According to an embodiment of the present application, the surface of the bio-cellulose film formed in the longitudinal direction or in the width direction is a surface where the foam is bonded to the bio-cellulose film.

Referring to FIG. 2B, a side view of the bio-cellulose film 12 is shown, wherein the bio-cellulose film 12 also has a plurality of backbone fibers 121a that are parallel to each other or extend in the longitudinal or width direction of the bio-cellulose film 12, and the interlayered fiber 121b interwoven with the backbone fibers 121.

The foam in the present application is not limited, as long as it is an open cell and biocompatible foam. For example, Polyurethane foam, polyvinyl alcohol foam, polyether foam, polyester foam, polylactic acid foam, polyolefin foam, chitosan foam, cellulose foam, alginate foam, gelatin foam and collagen foam.

The foam is made of a single material or is made of a plurality of materials to form a foam having a composite structure. Further, the foam may have a multilayer structure, which refers to the layers each are continuous and the same in material, but have different physical properties such as density, porosity, permeability, compression ratio, etc. The physical properties can be obtained by adjusting various parameters during the manufacturing process. Also, the multilayer structure may refer to the layers each are different in materials, for example, a layered structure of polyurethane and polyvinyl alcohol. The layers each in the multilayer structure can provide different functions as desired, such as contact layers, absorption layers and drainage layers.

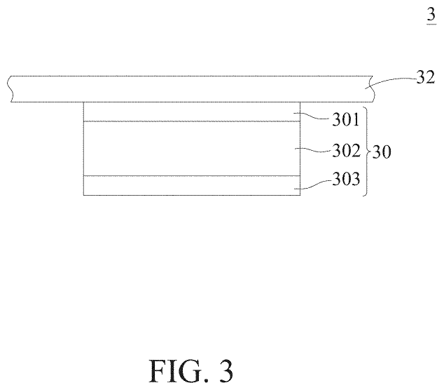

The foam of the negative pressure wound dressing in the present application can be a single layer structure, besides, in an embodiment as shown in FIG. 3, the negative pressure wound dressing 3 in the present application includes a foam 30 having a multilayer structure and the bio-cellulose film 32. The multilayer structure sequentially includes a contact layer 301, an absorption layer 302, and a drainage layer 303. The contact layer 301 contacts the bio-cellulose film 32. The contact layer 301 has larger pores than those pores formed in the absorption layer 302, so that parts of the bio-cellulose of the bio-cellulose film 32 can grow into the pores of the contact layer 301 to enhance the bonding between the bio-cellulose film 32 and the foam 30, and also the wound exudate can quickly enter the foam. The absorption layer 302 has better liquid absorption characteristics, and can temporarily accommodate the wound exudate. The drainage layer 303 has better exudate-transferring characteristics, such that the exudate absorbed by the foam can be drained to the liquid delivery tube of the negative pressure wound device.

After the bio-cellulose film is formed on and bond to the foam, the negative pressure wound dressing in the present application is formed. Then the obtained negative pressure wound dressing is washed to be neutral. The washing method is, for example, boiling with 1% of NaOH solution at 80° C. or higher for 15 minutes, then washed with water.

The washed negative wound dressing is then subjected to sterilization treatment. The sterilization treatment includes, but not limited to, autoclaving, gamma irradiation sterilization, ethylene oxide (EO) sterilization, and the like. Preferably, the autoclaving is used, for example, at 1.2 atm and 121° C., for 20 minutes.

The negative pressure wound dressing of the present application may further comprise a drug. In one embodiment, the drug can be added after the washing procedure. Since the bio-cellulose film and the foam both have pores, the drug can be kept in the bio-cellulose film, in the foam, or in both of them. For example, the drug for promoting wound healing, such as antibacterial, anti-inflammatory, and promoting cell growth drugs can be selected. The drug includes but is not limited to, at least one selected from the group consisting of antibiotics, antibacterial agents, growth factors, fibrin, vasodilators, angiogenesis promoters, antioxidants, and anti-inflammatory agents.

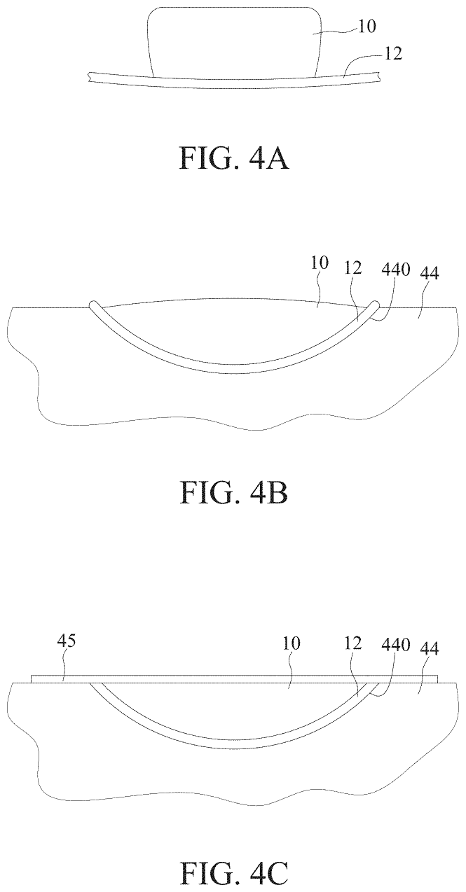

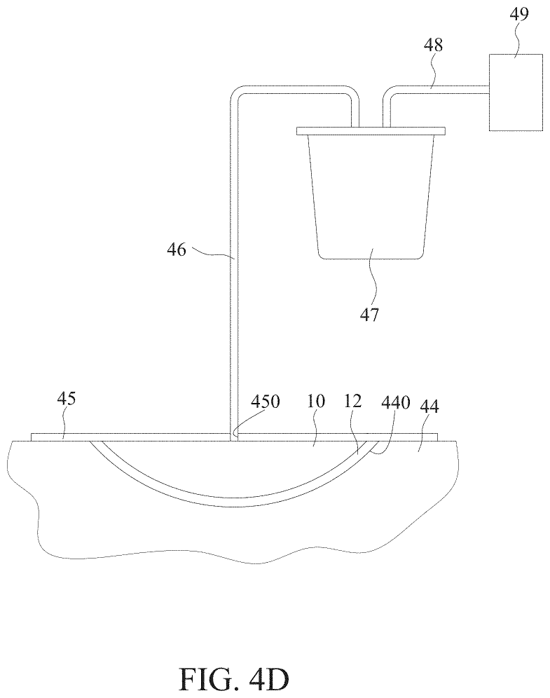

The negative pressure wound dressing of the present application can be applied to the negative pressure wound therapy (NPWT). In one embodiment, the negative pressure wound dressing and the sealing film, the liquid delivery tube, the liquid-collecting tank and the negative pressure source constitute a negative pressure wound treatment device. Specifically, the negative pressure wound treatment device of the present application can be implemented as shown in the schematic diagrams of FIG. 4A to 4G to treat a wound.

As shown in FIG. 4A, prepare the negative pressure wound dressing of the present application, which includes foam 10 and bio-cellulose film 12. Then, as shown in FIG. 4B, the negative pressure wound dressing of the present application is filled into the wound 440 of the injured site 44. The filling method is such that the bio-cellulose film 12 is directed toward the surface of the wound 440, and the foam 10 faces relatively outward.

The shape and volume of the foam 10 must be compatible with the wound 440. Before filling the foam 10 into the wound 440, the foam 10 can be appropriately trimmed so that the bio-cellulose film 12 is completely adhered to the surface of the wound 440 and the foam 10 is approximately flush with normal skin at injured site 44 outside the wound 440.

As shown in FIG. 4C, the sealing film 45 is completely covered with the negative pressure wound dressing. The area of the sealing film 45 needs to be larger than the wound 440, so that edges of the sealing film 45 can adhere to normal skin at injured site 44 outside the wound 440.

As shown in FIG. 4D, an opening 450 is formed in the center of the sealing film 45 and penetrates through two opposite surfaces of the sealing film 45, so that one end of the liquid delivery tube 46 can extend into the foam 10 of the negative pressure wound dressing through the opening 450. The other end of the liquid delivery tube 46 is connected to the liquid-collecting tank 47 and the liquid-collecting tank 47 is then connected to the negative pressure source 49, such as a pump through the connecting tube 48. After being sucked by the negative pressure source 49, pus and infectious substances in the wound 440 flow to the liquid-collecting tank 47 through the bio-cellulose film 12, the foam 10 and the liquid delivery tube 46.

Due to the barrier of the bio-cellulose film between the foam and the wound surface, wherein the bio-cellulose film has excellent anti-adhesion properties, therefore, the negative pressure wound dressing of the present application does not stick to the wound, and the new tissue does not grown into the foam, reducing the pain that the patient suffers when replacing the negative pressure wound dressing, and greatly decreasing the secondary damage to the wound. By leaving the drug in the pores of the foam and/or bio-cellulose film, the drug can be further released to the wound during the treatment, promoting the healing of the wound.

What is claimed is:

1. A negative pressure wound dressing comprising:
foam; and
a bio-cellulose film having a three-dimensional reticular structure, the bio-cellulose film being bonded to a surface of the foam, with a projected area of the bio-cellulose film on a surface of the foam bonded to the bio-cellulose film being greater than or equal to a projected area of the foam.

2. The negative pressure wound dressing of claim 1, wherein the bio-cellulose film is formed by bacteria of at least one genus selected from the group consisting of Gluconacetobacter, Acetobacter, Rhizobium, Sarcina, Pseudomonas, Achromobacter, Alcaligenes, Enterobacter, Azotobacter and Agrobacterium.

3. The negative pressure wound dressing of claim 1, wherein the three-dimensional reticular structure has a plurality of backbone fibers parallel to each other and a plurality of interlayered fibers interwoven with any two adjacent backbone fibers.

4. The negative pressure wound dressing of claim 3, wherein the backbone fibers and the interlayered fibers are made of bio-cellulose, and a diameter of the backbone fiber is greater than or equal to a diameter of the interlayered fiber.

5. The negative pressure wound dressing of claim 4, wherein the bio-cellulose has a diameter of 20 nm to 100 nm.

6. The negative pressure wound dressing of claim 1, wherein the bio-cellulose film has a bio-cellulose content of 0.0013 g/cm² to 0.0018 g/cm² per unit area.

7. The negative pressure wound dressing of claim 1, wherein the negative pressure wound dressing further comprises a drug included in at least one of the foam and the bio-cellulose film.

8. The negative pressure wound dressing of claim 7, wherein the drug is at least one selected from the group consisting of an antibiotic, an antibacterial agent, a growth factor, fibrin, a vasodilator, an angiogenesis promoter, an antioxidant and an anti-inflammatory agent.

9. The negative pressure wound dressing of claim 1, wherein the foam comprises at least one material selected from the group consisting of polyurethane foam, polyvinyl alcohol foam, polyether foam, polyester foam, polylactic acid foam, polyolefin foam, chitosan foam, cellulose foam, alginate foam, gelatin foam and collagen foam.

10. The negative pressure wound dressing of claim 1, wherein the foam has a single layer structure or a multilayer structure.

11. The negative pressure wound dressing of claim 10, wherein the multilayer structure comprises a contact layer, an absorption layer and a drainage layer in sequence, and wherein the contact layer is in contact with the bio-cellulose film.

12. A negative pressure wound treatment device, comprising:
the negative pressure wound dressing of claim 1;
a liquid delivery tube for connecting the negative pressure wound dressing; and
a negative pressure source.

13. The negative pressure wound treatment device of claim 12, further comprising a sealing film disposed on the negative pressure wound dressing for covering a wound and a normal skin outside the wound.

14. The negative pressure wound treatment device of claim 13, wherein an opening is formed in a center of the sealing film and penetrates through two opposite surfaces of the sealing film, and one end of the liquid delivery tube extends to the foam of the negative pressure wound dressing through the opening.

15. The negative pressure wound treatment device of claim 12, further comprising a liquid-collecting tank for connecting the other end of the liquid delivery tube, and the liquid-collecting tank is connected to the negative pressure source through a connecting tube.
